(19) United States
(12) Patent Application Publication
(10) Pub. No.: US 2004/0181971 A1
(43) Pub. Date: Sep. 23, 2004

(54) Footbed

(75) Inventors: Jay N. Turkbas, Orono, MN (US); Peter Daley, Yampa, CO (US); Steven J. Washburn, Shorewood, MN (US)

Correspondence Address: Schwegman, Lundberg, Woessner & Kluth, P.A., P.O. Box 2938, Minneapolis, MN, 55402, US

(73) Assignee: E-Z Gard Industries, Inc..

(21) Appl. No.: 10/606,480

(22) Filed: Jun. 26, 2003

Related U.S. Application Data
(60) Provisional application No. 60/456,387, filed on Mar. 21, 2003.

(51) Int. Cl. A43B 13/38; A61F 5/14

(52) U.S. Cl. 36/44; 36/140; 36/88

(57) ABSTRACT

A footbed for insertion into a shoe. The footbed includes an insole with a heel area, a metatarsal area and an arch area between the heel area and the metatarsal area. The insole also includes a lateral side and a medial side. A support is attached to the insole and extends between the arch area and the heel area of the insole. The support extends from the lateral side to the medial side of the insole to reduce pronation in a foot that is supported by the footbed.

### RELATED APPLICATION

This application claims the benefit of U.S. Provisional Application Serial No. 60/456,387 filed Mar. 21, 2003.

### TECHNICAL FIELD

The present invention relates generally to a footbed, and more particularly to a footbed that reduces pronation within a foot which is supported by the footbed.

### TECHNICAL BACKGROUND

A typical footbed supports one or more portions of a foot to minimize stress on weight bearing areas of the foot. Footbeds can be flat, contoured, or shaped to conform to a particular foot (i.e., customized). Conventional footbeds usually increase foot comfort but do not provide significant control of foot motion, especially with regard to the joints in a foot.

Motion of the joints in the foot causes stresses on the joints that can lead to pronation. Pronation is a complex foot motion which produces the partial collapse of the medial longitudinal arch of the foot. Excessive pronation is the source of many lower extremity pathologies, including muscle tiredness and inflammation, foot and knee joint pain, tendinitis, ligament strain, and even neurological damage.

The forces that are transmitted through an individual's feet during running, or some other athletic activity, can easily be multiple times an individual's body weight. Therefore, the ability of a footbed to restrict the motion of joints in a foot to minimize pronation is especially critical during athletic activity.

One drawback with conventional footbeds is that they do not sufficiently minimize pronation, especially during athletic activity. Since a significant percentage of the population suffers from the adverse effects of excessive pronation, there is a need for a durable footbed that alleviates pronation.

### SUMMARY OF THE INVENTION

A footbed for insertion into a shoe. The footbed includes an insole with a heel area, a metatarsal area and an arch area between the heel area and the metatarsal area. The insole also includes a lateral side and a medial side. A support is attached to the insole and extends between the arch area and the heel area of the insole. The support also extends from the lateral side to the medial side of the insole to reduce pronation in a foot that is supported by the footbed.

In another embodiment, a footbed for insertion into a shoe includes an insole with a heel impact area, a metatarsal area and an arch area between the heel impact area and the metatarsal area. The insole also includes a lateral side and a medial side. A support is attached to the insole and surrounds the heel impact area. The support also extends from the heel impact area toward the metatarsal area along the medial side of the insole.

In still another embodiment, a footbed for insertion into a shoe includes an insole with a heel area, a metatarsal area and an arch area between the heel area and the metatarsal area. The insole also includes a lateral side and a medial side. A support is attached to the insole and extends between the arch area and the heel area of the insole from the lateral side to the medial side of the insole. The support includes a lateral end, a medial end and a center section between the lateral end and the medial end. The lateral and medial ends of the support are wider than the center section.

In yet another embodiment, a footbed for insertion into a shoe includes an insole having a heel area with a heel impact area, a metatarsal area with a first metatarsal area and an arch area between the heel area and the metatarsal area. The insole also includes a lateral side and a medial side. A support is embedded in the insole and extends between the arch area and the heel area from the lateral side to the medial side of the insole. The support also surrounds the heel impact area and extends from the heel area to the first metatarsal area along the medial side of the insole.

Other features will become obvious with a reading of the following specification and appended claims as well as a review of the figures.

### BRIEF DESCRIPTION OF THE DRAWINGS

The present invention will be more fully understood, and further features will become apparent, when reference is made to the following detailed description and the accompanying drawings. The drawings are merely representative and are not intended to limit the scope of the claims. Like parts depicted in the drawings are referred to by the same reference numerals.

FIG. 1 is a plan view of an example insole.

FIG. 2 is a plan view of an example footbed.

FIG. 3 is a section view taken along lines 3-3 in FIG. 2.

FIG. 4 is a section view taken along lines 4-4 in FIG. 2.

FIG. 5 is a section view taken along lines 5-5 in FIG. 2.

FIG. 6 is a plan view of another footbed.

FIG. 7 is a section view taken along lines 7-7 in FIG. 6.

FIG. 8 is a section view taken along lines 8-8 in FIG. 6.

### DETAILED SPECIFICATION

In the following detailed description, reference is made to the accompanying drawings which show by way of illustration specific embodiments in which the invention may be practiced. These embodiments are described in sufficient detail to enable those skilled in the art to practice the invention. It is to be understood that other embodiments may be utilized and that structural changes made such that the following detailed description is not to be taken in a limiting sense.

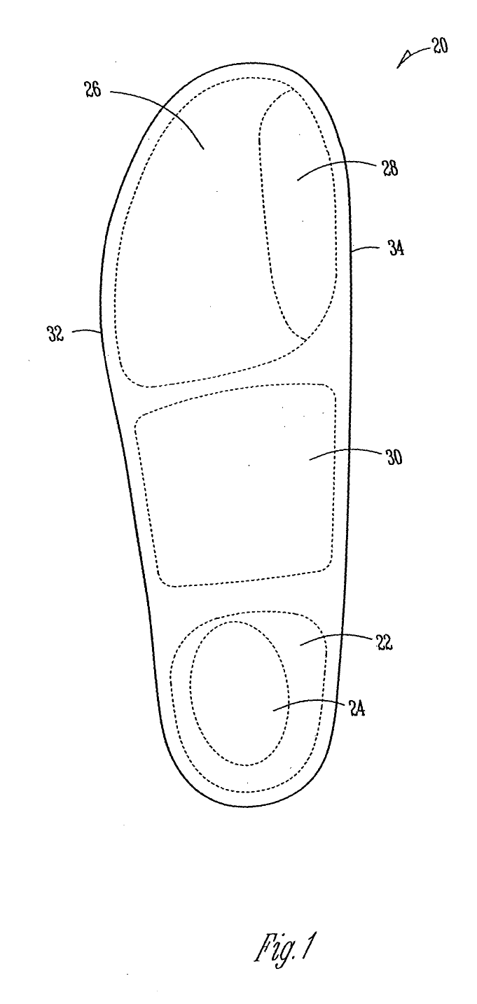

FIG. 1 illustrates an example insole 20 that includes a heel area 22 having a heel impact area 24, a metatarsal area 26 having a first metatarsal area 28, and an arch area 30 between the heel area 22 and the metatarsal area 26. Insole 20 also includes a lateral side 32 and a medial side 34. Although the insoles 20 described hereafter may take different forms in other embodiments, each of the insoles 20 includes areas and sides as generally described with reference to FIG. 1.

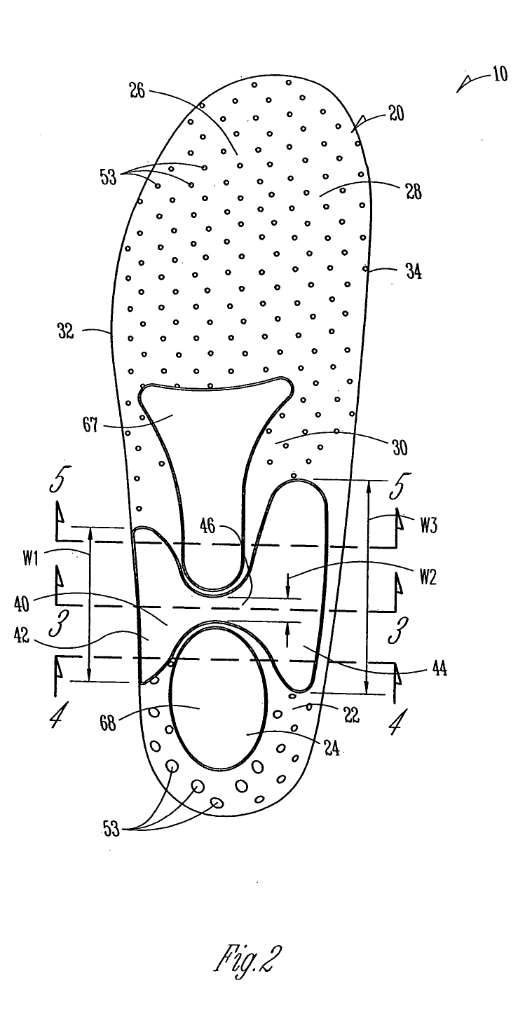

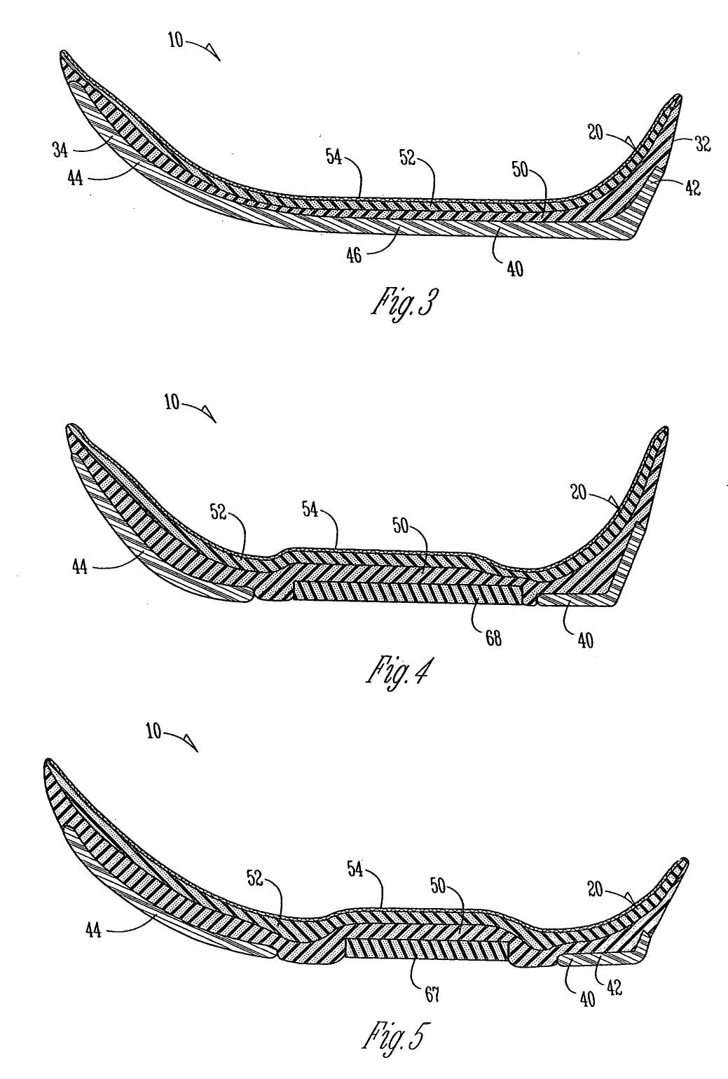

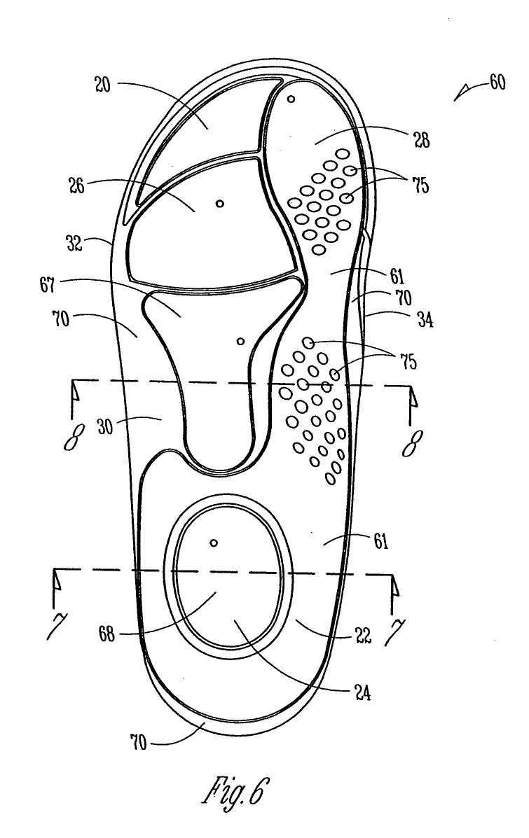

FIGS. 2-5 illustrate a footbed 10 for insertion into a shoe. Footbed 10 includes an insole 20 and a support 40 that is attached to insole 20. Support 40 extends between the arch area 30 and the heel area 22 of the insole 20, and from a lateral side 32 to a medial side 34 of the insole 20.

The support 40 includes a lateral end 42, a medial end 44 and a center section 46 between the lateral end 42 and the medial end 44. In the illustrated example embodiment, the lateral and medial ends 42, 44 are wider than the center section 46 (i.e., compare dimension W1 with dimensions W2, W3 in FIG. 2).

It should be noted one or more portions of insole 20 and/or support 40 may be flat or contoured. As used herein, contoured means that the insole is shaped to fit one or more portions of a foot that engages the footbed 10 (see, e.g., contoured shape of insole 20 in FIGS. 3-5).

In the illustrated example embodiment, support 40 is embedded in the insole 20 (see FIG. 3). It should be noted that none, some or all of the support 40 may be embedded in insole 20.

The sample insole 20 of FIGS. 2-5 includes a first layer 50 and a second layer 52 such that the support 40 is embedded in the first layer 50. In the example embodiment illustrated in FIG. 2, second layer 52 extends through first layer 50 at one or more portions 53 on first layer 50. It should be noted that the size, number, location and shape of portions 53 may be varied in other embodiments.

In one example embodiment, first layer 50 may be a formed of a cushioning material that is able to withstand the friction forces generated between footbed 10 and a shoe when a foot is engaged with footbed 10 inside the shoe. One example material for first layer may 50 is EVA resin foam, although other materials may be used instead.

In addition, second layer 52 may be formed of even greater cushioning material. Second layer 52 may be EVA foam, although other materials may be used instead.

Support 40 may be formed from an impact-resistant material that reduces the effects of any impact forces which are generated on support 40, such as by a foot and a shoe during athletic activity. Support 40 may be a thermoplastic urethane material, although other materials may be used instead.

In the sample embodiment illustrated in FIGS. 3-5, the insole 20 includes a third layer 54 that covers second layer 52. Third layer may 54 formed from a woven material that is adapted to provide comfort to a foot that engages footbed 10. Third layer 54 may be a polyester fabric, although other materials may be used instead.

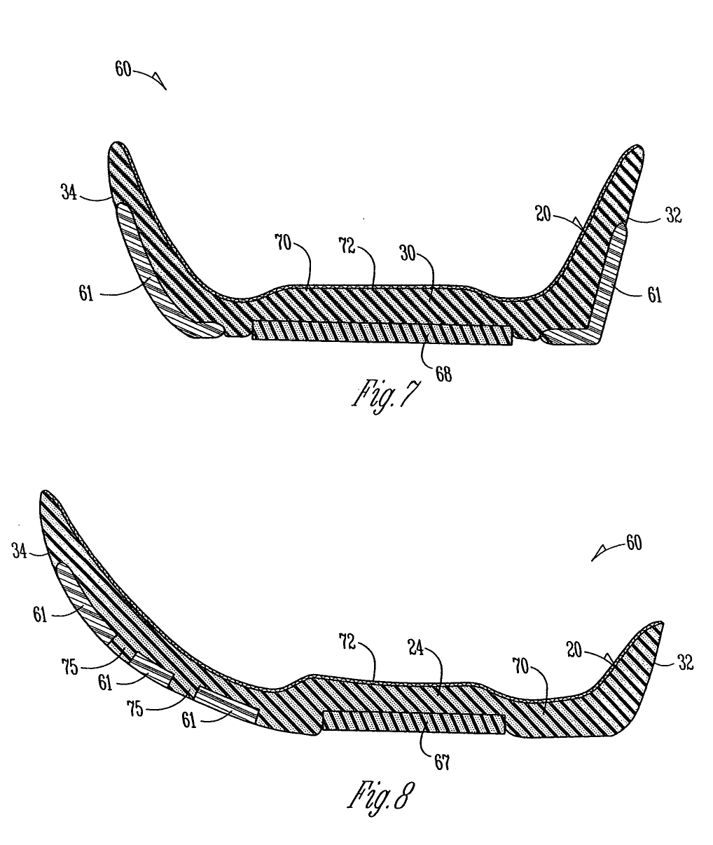

FIGS. 6-8 illustrate another example footbed 60 for insertion into a shoe. Footbed 60 includes an insole 20 with general areas and sides as discussed above with regard to FIG. 1. A support 61 is attached to insole 20 and surrounds the heel impact area 24. The support 61 extends toward the metatarsal area 26 of the insole 20 along the medial side 34 of the insole 20. In the sample embodiment illustrated in FIG. 6, support 61 extends along the medial side 34 of insole 30 to the first metatarsal area 28 of insole 20.

The sample insole 20 of FIGS. 6-8 includes a first layer 70 and a second layer 72 such that support 61 is embedded in insole 20. Although support 61 is shown as being entirely embedded in first layer 70 of insole 20, none or some of support 61 may be embedded in first layer 70 and/or insole 20 in other embodiments.

In one example embodiment, first layer 70 may be a formed of a cushioning material that is able to withstand the friction forces generated between footbed 10 and a shoe when a foot is engaged with footbed 10 inside the shoe. One example material for first layer may 70 is EVA foam, although other materials may be used instead.

Second layer may 72 formed from a woven material that is adapted to provide comfort to a foot that engages footbed 10. Second layer 72 may be polyester fabric, synthetic leather or leather, although other materials may be used instead.

In addition, support 61 may be formed from an impact-resistant material that reduces the effects of any impact forces which are generated on support 61, such as by a foot and a shoe during athletic activity. Support 61 may be thermoplastic urethane, although other materials may be used instead.

As shown in FIGS. 6 and 8, at least a portion of insole 20 may extend through support 61. In the illustrated example embodiment, first layer 70 of insole 20 extends through support 61 at one or more locations 75 on the medial side 34 of insole 20.

As shown in FIGS. 2, 5, 6 and 8, the sample footbeds 10, 60 include an arch pad 67 that is attached to the arch area 30 of insole 20 adjacent to respective supports 40, 61. In addition, as shown in FIGS. 2, 4, 6 and 7, the sample footbeds 10, 60 include a heel pad 68 that is attached to the heel impact area 24 of insole 20 adjacent to respective supports 40, 61.

In the example embodiments illustrated in FIGS. 2-8, arch pad 67 and heel pad 68 are embedded in insole 20. It should be noted that none, some or all of the arch pad 67 and/or heel pad 68 may be embedded in insole 20. In addition, arch pad 67 and heel pad 68 may be EVA rubber, EVA or rubber (among other materials).

The present invention may be embodied in other specific forms without departing from the scope of the present invention; therefore, the illustrated embodiments should be considered in all respects as illustrative and not restrictive, reference being made to the appended claims rather than to the foregoing description to indicate the scope of the invention.

We claim:

1. A footbed for insertion into a shoe, the footbed comprising:
an insole that includes a heel area, a metatarsal area and an arch area between the heel area and the metatarsal area, the insole including a lateral side and a medial side; and
a support attached to the insole, the support extending between the arch area and the heel area of the insole, and the support extending from the lateral side to the medial side of the insole.

2. The footbed of claim 1, wherein the support includes a lateral end, a medial end and a center section between the lateral end and the medial end, the lateral and medial ends being wider than the center section.

3. The footbed of claim 1, wherein the insole is contoured.

4. The footbed of claim 1, wherein the support is embedded in the insole.

5. The footbed of claim 1, wherein the insole includes a first layer and a second layer such that the support is attached to the first layer.

6. The footbed of claim 5, wherein the first layer is EVA resin foam, the second layer is EVA foam and the support is a thermoplastic urethane.

7. The footbed of claim 6, further comprising a third layer covering the second layer, wherein the third layer is a polyester fabric.

8. The footbed of claim 5, wherein at least a portion of the second layer extends through the first layer.

9. The footbed of claim 1, wherein the support extends from the heel area to the metatarsal area along the medial side of the insole.

10. A footbed for insertion into a shoe, the footbed comprising:
an insole that includes a heel impact area, a metatarsal area and an arch area between the heel impact area and the metatarsal area, the insole including a lateral side and a medial side; and
a support attached to the insole, the support surrounding the heel impact area and extending from the heel impact area toward the metatarsal area along the medial side of the insole.

11. The footbed of claim 10, wherein the metatarsal area includes a first metatarsal area such that the support extends along the medial side of the insole to the first metatarsal area.

12. The footbed of claim 10, further comprising an arch pad attached to the arch area of the insole adjacent to the support.

13. The footbed of claim 12, wherein the arch pad is embedded in the insole.

14. The footbed of claim 10, further comprising a heel pad attached to the heel impact area of the insole adjacent to the support.

15. The footbed of claim 14, wherein the heel pad is embedded in the insole.

16. The footbed of claim 10, wherein the insole includes a first layer and a second layer and the support is attached to the first layer.

17. The footbed of claim 10, wherein the support is embedded in the insole.

18. The footbed of claim 17, wherein at least a portion of the insole extends through the support.

19. The footbed of claim 18, wherein the portion of the insole that extends through the support extends through the support on the medial side of the insole.

20. A footbed for insertion into a shoe, the footbed comprising:
an insole that includes a heel area, a metatarsal area and an arch area between the heel area and the metatarsal area, the insole including a lateral side and a medial side; and
a support attached to the insole, the support extending between the arch area and the heel area of the insole from the lateral side to the medial side of the insole, the support including a lateral end, a medial end and a center section between the lateral end and the medial end, the lateral and medial ends being wider than the center section.

21. The footbed of claim 20, wherein the support is embedded in the insole.

22. The footbed of claim 20, wherein the insole includes a first layer and a second layer such that the support is attached to the first layer.

23. A footbed for insertion into a shoe, the footbed comprising:
an insole that includes a heel area with a heel impact area, a metatarsal area with a first metatarsal area and an arch area between the heel area and the metatarsal area, the insole including a lateral side and a medial side; and
a support embedded in the insole, the support extending between the arch area and the heel area, the support extending from the lateral side to the medial side of the insole, the support surrounding the heel impact area and extending from the heel area to the first metatarsal area along the medial side of the insole.

24. The footbed of claim 23, further comprising:
an arch pad embedded in the arch area of the insole adjacent to the support; and
a heel pad embedded in the heel impact area of the insole adjacent to the support.

25. The footbed of claim 23, wherein at least a portion of the insole extends through the support on the medial side of the insole.
